(19) United States
(12) Patent Application Publication
(10) Pub. No.: US 2024/0050955 A1
(43) Pub. Date: Feb. 15, 2024

(54) MULTI-FUNCTIONAL INTEGRATED MOLECULAR TEST CARTRIDGE AND APPLICATION THEREOF

(71) Applicant: Ustar Biotechnologies (Hangzhou) Ltd., Hangzhou (CN)

(72) Inventors: Yuankui Lin, Hangzhou (CN); Qimin You, Hangzhou (CN); Wendy Zang, Hangzhou (CN); Yanqiong Zhou, Hangzhou (CN); Xiangyu Zeng, Hangzhou (CN); Xiangyu Yang, Hangzhou (CN); Zhaocheng Hu, Hangzhou (CN); Junwei Yu, Hangzhou (CN); Jinxiao Shuai, Hangzhou (CN); Yizhi Lin, Hangzhou (CN)

(21) Appl. No.: 18/479,132

(22) Filed: Oct. 2, 2023

(30) Foreign Application Priority Data
Mar. 3, 2023 (CN) 202310195175.1

(51) Int. Cl. B01L 3/00

(52) U.S. Cl. CPC B01L 3/523; B01L 2300/044; B01L 2300/0672; B01L 2200/16; B01L 2200/0689

(57) ABSTRACT

Provided is a multi-functional integrated molecular test cartridge and application thereof. The multi-functional integrated molecular test cartridge includes a deformable assembly, a blocking assembly and a reaction assembly, where the blocking assembly is placed between the deformable assembly and the reaction assembly, and a channel in communication with the reaction assembly and the deformable assembly is formed on the blocking assembly. The multi-functional integrated molecular test cartridge can be used for isothermal amplification reactions such as cross priming amplification (CPA), recombinase polymerase amplification (RPA), loop-mediated isothermal amplification (LAMP) and rolling circle amplification (RCA), can achieve integrated pre-loading of all reagents, a whole-process integrated test from a sample to a result, and flexible sample processing modes, and has high biological safety and a low risk of product and sample leakage.

### REFERENCE TO SEQUENCE LISTING

The substitute sequence listing is submitted as a XML file filed via EFS-Web, with a file name of “Substitute_Sequence_Listing HZHC-USP1233227. XML”, a creation date of Dec. 9, 2023, and a size of 27,358 bytes. The substitute sequence Listing filed via EFS-Web is a part of the specification and is incorporated in its entirety by reference herein.

### TECHNICAL FIELD

The present disclosure relates to the field of molecular tests, and particularly to a multi-functional integrated molecular test cartridge and application thereof.

### DESCRIPTION OF RELATED ART

Nucleic acid test, a vital branch of molecular diagnosis, is used to detect a pathogen or a specific gene by specifically amplifying a nucleic acid fragment of a test target. The current prevailing method of the nucleic acid test is polymerase chain reaction (PCR). A common test process of polymerase chain reaction includes sample processing, nucleic acid extraction, PCR amplification, and amplified product detection. This process requires various types of consumables, and a pipette frequently used for liquid transfer. Accordingly, the nucleic acid test generally requires a specialized laboratory and auxiliary tool, and further has high requirements for professional skills of an operator. Such complex operations not only increase test time and workload, but also tend to cause environmental pollution, resulting in a deviation of a test result.

Point-of-care testing (POCT) is a novel method arising in recent years, which allows immediate analysis at a sampling site and omits complex processing procedures of a laboratory test for a sample, thereby rapidly obtaining a test result. However, some POCT-type molecular diagnostic reagents employ microfluidics or integrated test cartridges. Despite an integration of the whole test process from samples to results, they are still complex in structure and high in process cost. Thus, the POCT-type molecular diagnostic reagents fail in popularization and application to the market. In addition, since POCT-type test consumables on the current market are rarely compatible with a variety of reagent fixation methods, a specific POCT-type test consumable adapted to a specific reagent is required, which increases the cost of the POCT-type molecular diagnostic reagents virtually.

### SUMMARY

Examples of the present disclosure provide a multi-functional integrated molecular test cartridge and application thereof. Multiple functions such as sample processing, reagent pre-loading, and nucleic acid amplification test can be achieved in the same test cartridge, and operation steps of a molecular test are simple and feasible such that a user can carry out both at-home test and mobile test in a field.

In a first aspect, an example of the present disclosure provides a multi-functional integrated molecular test cartridge. The multi-functional integrated molecular test cartridge includes a deformable assembly, a blocking assembly and a reaction assembly, where the blocking assembly is placed between the deformable assembly and the reaction assembly; a breakable assembly is arranged on the blocking assembly, and the breakable assembly is switched from a sealed state to a broken state under action of an external force; when the breakable assembly is in the sealed state, the blocking assembly blocks communication between the reaction assembly and the deformable assembly; and when the breakable assembly is in the broken state, the breakable assembly is separated from the blocking assembly, and a channel in communication with the reaction assembly and the deformable assembly is formed on the blocking assembly.

In a second aspect, an example of the present disclosure provides a multi-functional integrated molecular test system. The multi-functional integrated molecular test system includes a multi-functional integrated molecular test cartridge and an incubator matching the test cartridge, where the multi-functional integrated molecular test cartridge is subjected to any one of high-temperature heating, vibration, ultrasound and amplification on the incubator.

In a third aspect, an example of the present disclosure provides a test method of a multi-functional integrated molecular test cartridge, which carries out a molecular test by the multi-functional integrated molecular test cartridge. The test method includes: pre-loading a multi-purpose buffer into a reaction assembly, and pre-loading a reaction reagent into a deformable assembly; adding a test sample into the reaction assembly, such that the test sample is lysed by the multi-purpose buffer; after the test sample is completely lysed, applying an external force to the deformable assembly to switch a breakable assembly to a broken state; applying an external force repeatedly to the deformable assembly or inverting the test cartridge, such that reagents in the reaction assembly and the deformable assembly are mixed; and after the reagents are fully mixed, placing the test cartridge upright, such that all liquid flows to a bottom of the reaction assembly, and placing the reaction assembly in an incubator for heating and an amplification reaction.

In a forth aspect, an example of the present disclosure provides a test method of a multi-functional integrated molecular test cartridge, which carries out a molecular test by the multi-functional integrated molecular test cartridge. The test method includes: pre-loading a multi-purpose buffer into a reaction assembly, and pre-loading a reaction reagent into a deformable assembly; applying an external force to the deformable assembly to switch a breakable assembly to a broken state; applying an external force repeatedly to the deformable assembly or inverting the test cartridge, such that reagents in the reaction assembly and the deformable assembly are mixed; and after the reagents are fully mixed, placing the test cartridge upright, such that all liquid flows to a bottom of the reaction assembly, adding a test object into the reaction assembly, and placing the reaction assembly in an incubator for amplification.

Main contributions and innovations of the present disclosure are as follows:

Examples of the present disclosure provide a multi-functional integrated molecular test cartridge and application thereof, a blocking assembly is arranged in the multi-functional integrated molecular test cartridge to achieve dry-wet separation of a reaction liquid and a dry material, such that storage stability and flexibility of the multi-functional integrated molecular test cartridge are enhanced; and a user can damage the blocking assembly through a simple operation to mix the dry material and the reaction liquid, such that multiple functions such as sample processing, reagent pre-loading and nucleic acid amplification test can be achieved in the same test cartridge, and an integrated test for complex samples such as sputum, a bronchoalveolar lavage fluid, pus and blood can be achieved. In some examples, a multi-purpose buffer that can process complex samples and is used for amplification reactions is pre-loaded therein, and by using the multi-purpose buffer, a structural design of the test cartridge is greatly simplified, such that it is not required to additionally and independently arrange a sample processing buffer and a reaction buffer in the test cartridge, thereby greatly simplifying the structural design of the test cartridge; and moreover, the multi-functional integrated molecular test cartridge provided in the solution is low in cost, simple in structure and user-friendly such that a user can carry out both at-home test and mobile test out of doors by the multi-functional integrated molecular test cartridge, and application scenarios of the molecular test are extremely expanded.

Details of one or more examples of the present disclosure will be provided in the following accompanying drawings and description, such that other features, objectives and advantages of the present disclosure are more clear and easy to understand.

### BRIEF DESCRIPTION OF THE DRAWINGS

The accompanying drawings described herein serve to provide further understanding and form a part of the present disclosure, and the illustrative examples and descriptions thereof serve to explain the present disclosure and are not to be construed as unduly limiting the present disclosure. In figures:

FIG. 1 is an overall schematic diagram of a multi-functional integrated molecular test cartridge according to an example of the present disclosure;

FIG. 2 is a schematic diagram of an internal structure of a multi-functional integrated molecular test cartridge according to an example of the present disclosure;

FIG. 3 is a schematic diagram of a broken state of a breakable assembly 40 of a multi-functional integrated molecular test cartridge according to an example of the present disclosure; and

FIG. 4 is a schematic diagram of a working state of a multi-functional integrated molecular test cartridge on an incubator according to an example of the present disclosure.

In the figures, deformable assembly 10, material storage area 11, first connecting area 12, blocking assembly 20, reaction assembly 30, second connecting area 31, reaction chamber 32 and breakable assembly 40.

### DETAILED DESCRIPTION OF THE EMBODIMENTS

Illustrative examples will be described in detail herein and shown in accompanying drawings. When the following description relates to the accompanying drawings, unless otherwise specified, the same numeral in different accompanying drawings denotes the same or similar elements. Embodiments described in the following illustrative examples do not denote all embodiments consistent with one or more examples of the description. On the contrary, the examples are merely instances of a device and a method consistent with some aspects of one or more examples of the description as detailed in the appended claims.

It should be noted that in other examples, steps of corresponding methods are not certainly executed in orders shown and described in the description. In some other examples, the method can include more or less steps than those described in the description. In addition, a single step herein may be decomposed into a plurality of steps for description in other examples. Moreover, a plurality of steps herein may be combined into a single step for description in other examples.

### EXAMPLE ONE

The solution provides a multi-functional integrated molecular test cartridge. The multi-functional integrated molecular test cartridge can be used for polymerase chain reaction (PCR), and isothermal amplification reactions such as cross priming amplification (CPA), recombinase polymerase amplification (RPA), loop-mediated isothermal amplification (LAMP) and rolling circle amplification (RCA), can achieve integrated pre-loading of all reagents, a whole-process integrated test from a sample to a result, and flexible sample processing modes, and has high biological safety and a low risk of product and sample leakage. Therefore, the multi-functional integrated molecular test cartridge is a novel test device that is extremely suitable for a user at home, or in a primary health unit and a field.

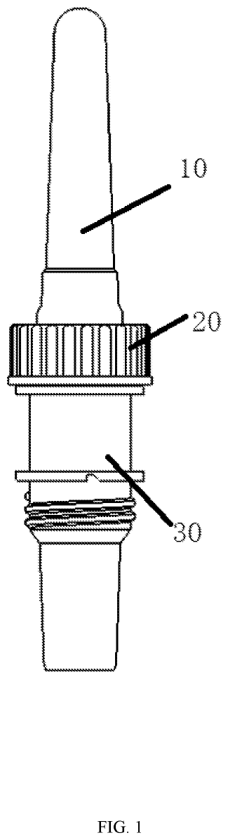

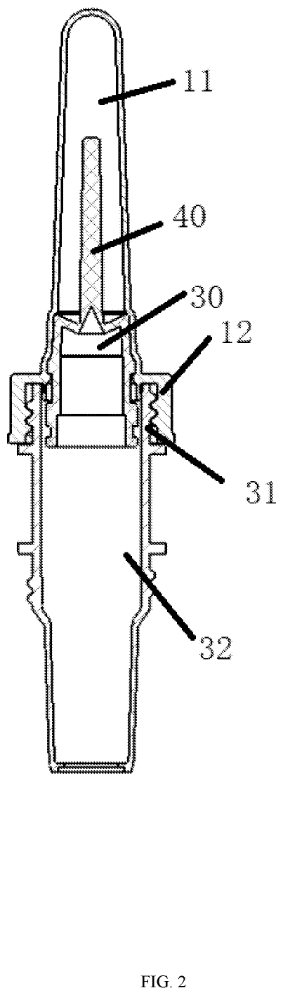

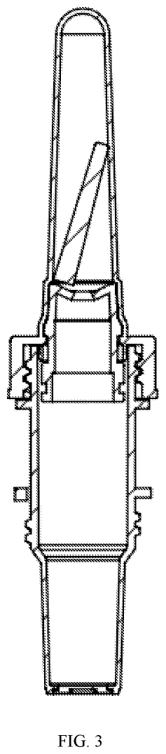

As shown in FIGS. 1-3, the multi-functional integrated molecular test cartridge provided in the solution includes a deformable assembly 10, a blocking assembly 20 and a reaction assembly 30, where the blocking assembly 20 is placed between the deformable assembly 10 and the reaction assembly 30; a breakable assembly 40 is arranged on the blocking assembly 20, and the breakable assembly 40 is switched from a sealed state to a broken state under action of an external force; when the breakable assembly 40 is in the sealed state, the blocking assembly 20 blocks communication between the reaction assembly 30 and the deformable assembly 10; and when the breakable assembly 40 is in the broken state, the breakable assembly 40 is separated from the blocking assembly 20, and a channel in communication with the reaction assembly 30 and the deformable assembly 10 is formed on the blocking assembly 20. The channel may be pre-arranged on the blocking assembly 20 or may be formed when the breakable assembly 40 is broken.

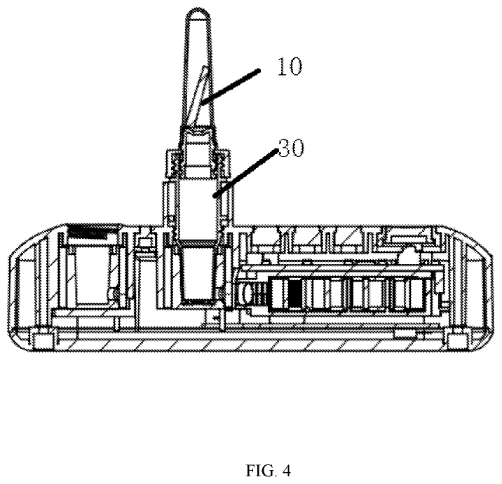

As shown in FIG. 4, the multi-functional integrated molecular test cartridge matches an incubator (not numbered in the figure) for use, an accommodation position for accommodating the reaction assembly 30 is provided on the incubator, and the reaction assembly 30 is inserted into the incubator for any one of high-temperature heating, vibration, ultrasound and amplification. In other words, the solution provides a multi-functional integrated molecular test system. The multi-functional integrated molecular test system includes the multi-functional integrated molecular test cartridge provided in the solution and an incubator matching the test cartridge, where the multi-functional integrated molecular test cartridge is subjected to any one of high-temperature heating, vibration, ultrasound and amplification on the incubator.

Specifically, a material storage area 11 is provided in the deformable assembly 10 of the multi-functional integrated molecular test cartridge provided in the solution, and a side of the material storage area 11 is attached to the blocking assembly 20, such that a material in the material storage area 11 is sealed and isolated. In other words, the blocking assembly 20 and an outer wall of the deformable assembly 10 delimit the material storage area 11 for hermetically storing the material. In an example of the solution, a liquid or solid reaction reagent is pre-loaded into the material storage area 11. The liquid reaction reagent may be, but is not limited to, an amplification reaction buffer, a sample lysis buffer, a sample diluent, or a sample preservation solution. The solid reaction reagent may be, but is not limited to, a primer, a probe, a reaction enzyme, a monovalent ion, a divalent ion, or an acid-base buffer, etc.

It should be noted that in an example of the solution, the deformable assembly 10 is made of a soft material, and the soft material is selected from one of plastic or polymer materials such as polyethylene (PE), polypropylene (PP) and acrylonitrile butadiene styrene (ABS). In other words, the deformable assembly 10 is made of plastic or polymer materials such as PE, PP and ABS through injection molding or three-dimensional (3D) printing, can be pressed and is soft in material. When the deformable assembly 10 is pressed by an external force, the deformable assembly 10 is deformed. Moreover, the deformable assembly 10 is deformed to apply an external force to the breakable assembly 40, such that the breakable assembly is switched between the sealed state and the broken state.

In some examples, the deformable assembly 10 is in a shape of an inverted U-shaped test cartridge. Particularly, an opening section of the deformable assembly 10 increases from the material storage area 11 to the blocking assembly 20, which allows the liquid or solid reaction reagent pre-loaded into the material storage area 11 to be better gathered at an end of the U-shaped deformable assembly 10.

In addition, for a purpose of addition of the reaction reagent, the reaction assembly 30 and the deformable assembly 10 are detachably connected. In some examples, a first connecting area 13 is provided on an end side of the deformable assembly 10 close to the reaction assembly 30, a second connecting area 31 is provided on an end side of the reaction assembly 30 close to the deformable assembly 10 correspondingly, and the first connecting area 13 and the second connecting area 31 match each other, such that the deformable assembly 10 and the reaction assembly 30 are detachably connected.

In some particular examples, the first connecting area 13 of the deformable assembly 10 is a threaded area with threads, and the second connecting area 31 of the reaction assembly 30 is a threaded area with corresponding threads. The threads on the first connecting area 13 are internal threads, and the threads on the second connecting area 31 are external threads correspondingly; alternatively, the threads on the first connecting area 13 are external threads, and the threads on the second connecting area 31 are internal threads correspondingly.

In some other particular examples, the first connecting area 13 of the deformable assembly 10 is a snap-fit bar, and the second connecting area 31 of the reaction assembly 30 is a snap-fit groove corresponding to the snap-fit bar; alternatively, the first connecting area 13 of the deformable assembly 10 is a snap-fit groove, and the second connecting area 31 is a snap-fit bar. Methods for connection between the first connecting area 13 and the second connecting area 31 include threaded connection, snap-fit connection, magnetic connection, etc.

The blocking assembly 20 provided in the solution is assembled at a position of an opening of the deformable assembly 10 to seal the material storage area. In some examples, in order to strengthen the connection between the blocking assembly 20 and the deformable assembly 10 to ensure a seal effect of the deformable assembly 10, a clamping rib is arranged at a side edge of the blocking assembly 20, and the clamping rib may be an annular protrusion or in other shapes.

In some examples, when the deformable assembly 10 is connected to the reaction assembly 30, the blocking assembly 20 is placed between the deformable assembly 10 and the reaction assembly 30, which ensures a relatively sealed state of the molecular test cartridge.

In the example of the solution, the blocking assembly 20 and the breakable assembly 40 match each other to achieve or block the communication between the reaction assembly 30 and the deformable assembly 10. When the breakable assembly 40 is in the broken state, the deformable assembly 10 and the reaction assembly 30 are in communication with each other.

Specifically, the solution provides two embodiments as follows:

Embodiment 1: a channel penetrating the reaction assembly 30 and the deformable assembly 10 is arranged on the blocking assembly 20, the breakable assembly 40 is arranged at a position of a through hole to block the channel, and when the breakable assembly 40 is separated from the blocking assembly 20, the channel on the blocking assembly 20 is exposed, such that the reaction assembly 30 and the deformable assembly 10 are in communication with each other.

In this case, a main body of the breakable assembly 40 is placed in the deformable assembly 10, and an end side of the main body blocks the channel of the blocking assembly 20.When being deformed relative to the blocking assembly 20, the deformable assembly 10 applies an external force to the breakable assembly 40, and the breakable assembly 40 is separated from the blocking assembly 20 under the action of the external force to expose the through hole on the blocking assembly 20.

In a particular example, the main body of the breakable assembly 40 is a hard-material member, and a joint between the breakable assembly 40 and the blocking assembly 20 is a soft-material member. A material of the soft-material member is easier to break than that of the hard-material member. A channel in communication with the deformable assembly 10 and the reaction assembly 30 is arranged in the blocking assembly 20. When the breakable assembly 40 is in the sealed state, a bottom of the breakable assembly 40 is connected to the blocking assembly 20, such that the channel in the blocking assembly 20 is blocked. When the breakable assembly 40 is subjected to an external force, the joint is separated from the blocking assembly 20, such that the channel on the blocking assembly 20 is exposed.

Specifically, both the hard-material member and the soft-material member may be made of plastic materials, and the soft-material member is thinner than the hard-material member such that the soft-material member may be damaged under the action of the external force. Damage methods include, but are not limited to, a method that a hole is formed between the breakable assembly 40 and the blocking assembly 20, or a method that the breakable assembly 40 and the blocking assembly 20 are completely separated from each other. In order to facilitate a user to break the breakable assembly 40, the hard-material member may be designed as a rod-like shape placed in the deformable assembly 10.

In addition, it should be noted that since the deformable assembly 10 is made of a soft material, the breakable assembly 40 placed in the deformable assembly 10 can be switched from the sealed state to the broken state due to deformation of the deformable assembly 10. In some examples, breakage methods for the breakable assembly 40 include, but are not limited to, bending the deformable assembly to one side, and pressing or twisting the deformable assembly. In a case that the breakable assembly 40 is broken by breaking the deformable assembly, the joint is tilted towards a side to be torn and separated from the blocking assembly 20, so as to expose the channel on the blocking assembly 20. In a case that the breakable assembly 40 is broken by pressing the deformable assembly, the joint is exploded under action of pressure and then separated from the blocking assembly 20. In a case that the breakable assembly 40 is broken by twisting the deformable assembly, the joint is twisted relative to the blocking assembly 20 to damage the structure of the joint, so as to be separated from the blocking assembly 20.

Embodiment 2: the blocking assembly 20 is made of a breakable material, the breakable assembly 40 is made of a rigid material, the breakable assembly 40 is connected to a top of the blocking assembly 20, and when the breakable assembly 40 is subjected to an external force, the breakable assembly 40 is broken from a bottom, and a channel is formed on the blocking assembly 20.

In this case, the main body of the breakable assembly 40 is placed in the deformable assembly 10, and a piercing member is arranged at a bottom of the main body. When being deformed relative to the blocking assembly 20, the deformable assembly 10 applies an external force to the breakable assembly 40, the breakable assembly 40 is separated from the blocking assembly 20 under the action of the external force, and the piercing member damages the blocking assembly 20, such that a channel in communication with the deformable assembly 10 and the reaction assembly 30 is formed.

In a particular example, the bottom of the breakable assembly 40 is a piercing member made of a hard material, when the breakable assembly 40 is in the sealed state, the piercing member is embedded into the blocking assembly 20, and when the breakable assembly 40 is in the broken state, the piercing member travels under the action of the external force, such that the channel in communication with the deformable assembly 10 and the reaction assembly 30 is formed on the blocking assembly 20. In this case, the blocking assembly 20 is made of a breakable plastic material, and the piercing member travels in the blocking assembly 20 in a process of being separated from the blocking assembly 20 under the action of the external force, such that the piercing member damages a sealing structure in the blocking assembly 20, and the channel in communication with the deformable assembly 10 and the reaction assembly 30 is formed.

In some examples, the blocking assembly 20 is made of a plastic material, and the breakable assembly 40 is made of plastic or polymer materials such as PE, PP and ABS through injection molding or 3D printing and has certain rigidity. The blocking assembly 20 may be of a cylindrical structure, and the breakable assembly 40 may be of a cylindrical structure.

Similarly, in some examples, breakage methods for the breakable assembly 40 include, but are not limited to, bending the deformable assembly to one side, and pressing or twisting the deformable assembly. In a case that the breakable assembly 40 is broken by breaking the deformable assembly, the piercing member is turned to one side to damage an internal structure of the blocking assembly 20. In a case that the breakable assembly 40 is broken by pressing the deformable assembly, the piercing member travels from bottom to top under action of pressure, such that a channel is formed in the blocking assembly 20. In a case that the breakable assembly 40 is broken by twisting the deformable assembly, the piercing member travels to be twisted upwards, such that a channel is formed in the blocking assembly.

A reaction chamber 32 is provided in the reaction assembly 30 according to the solution, and reactants are placed in the reaction chamber 32 for reactions. In order to enable the reaction assembly 30 to better match the incubator, a third connecting area 33 is provided on an outer peripheral side of the reaction assembly 30, and the third connecting area matches an accommodation position on the incubator. In some examples, the third connecting area 33 is external threads, and certainly, the third connecting area 33 may be a snap-fit or a snap groove.

The reaction assembly 30 in the solution is made of plastic or polymer materials such as PE, PP and ABS through injection molding or 3D printing, has certain rigidity, and can withstand high temperature. In addition, the reaction assembly 30 is made of a transparent material, which ensures fluorescence or visible light detection can be carried out on the reaction assembly by an external optical probe subsequently. Certainly, the material of the reaction assembly 30 may withstand vibration and ultrasound.

The multi-functional integrated molecular test cartridge provided in the solution can achieve dry-wet separation between a liquid reaction liquid and a solid material, and can further achieve separate storage of two different liquids. In some examples, in order to not independently arrange a sample processing buffer and a reaction buffer, the solution provides a multi-purpose buffer. The multi-purpose buffer may be used for lysing or processing a sample and used as a buffer in an amplification reaction system. Specifically, components of the multi-purpose buffer include: Tris-HCl at pH 6-9, dimethyl sulfoxide, TritonX 100, ethylenediaminetetraacetic acid (EDTA) disodium salt and dodecyl-β-D-maltoside. Tris-HCl acts as an acid-base buffer to stabilize a pH value of a system at 6-9. The dimethyl sulfoxide acts as auxiliary reagent for lysing the sample and an amplification reaction. TritonX 100 plays a role in lysing a protein coat or phospholipid bilayer membrane of the sample. The EDTA disodium salt plays a role in protecting the nucleic acid sample. Dodecyl-β-D-maltoside can degrade the biological phospholipid bilayer membrane and play a role in lysing the sample.

Specifically, in a particular example, the components of the multi-purpose buffer include 5 nm-500 nm Tris-HCl at pH 6-9, 1%-2% dimethyl sulfoxide, 0. 1%-10% TritonX 100, 0.1 mM-10 mM EDTA disodium salt, and 0. 1%-1% dodecyl-β-D-maltoside. Correspondingly, in the solution, the multi-purpose buffer is pre-loaded into the reaction assembly 30, and the reaction reagent is placed in the deformable assembly 10.

Certainly, in some other examples, a sample lysis buffer is pre-loaded into the reaction assembly 30, and a liquid or solid reaction reagent is pre-loaded into the deformable assembly 10. Moreover, the multi-purpose buffer or sample lysis buffer in the solution may be made into a dry material through vitrification, freeze-drying, volatilization-drying, etc. In this case, the multi-functional integrated molecular test cartridge provided in the solution can achieve the separate storage of dry and wet materials.

### EXAMPLE TWO

The solution provides a test method of a multi-functional integrated molecular test cartridge. The test method achieves a molecular test by the multi-functional integrated molecular test cartridge described in Example One, and includes steps as follows:
pre-load a multi-purpose buffer into a reaction assembly 30, and pre-load a reaction reagent into a deformable assembly 10;
add a test sample into the reaction assembly 30, such that the test sample is lysed by the multi-purpose buffer;
apply an external force to the deformable assembly 10 after the test sample is completely lysed to switch a breakable assembly 40 to a broken state;
apply an external force repeatedly to the deformable assembly 10 or invert the test cartridge, such that reagents in the reaction assembly 30 and the deformable assembly 10 are mixed; and
place the test cartridge upright after the reagents are fully mixed, such that all liquid flows to a bottom of the reaction assembly 30, and place the reaction assembly 30 in an incubator for heating and an amplification reaction.

In this way, a user is only required to add the test sample into the reaction assembly 30, and an integrated test for the test sample can be achieved without adding an additional reagent.

Specifically, when a user carries out a molecular test for a test sample by the multi-functional integrated molecular test cartridge, the multi-functional integrated molecular test cartridge is opened through rotation, the test sample is added into the reaction assembly and fully mixed with the multi-purpose buffer, and then the test cartridge is screwed and closed, such that the entire test cartridge is in a sealed state. In a sample lysis process, a chemical substance in the multi-purpose buffer and the test sample react at room temperature, or the bottom of the reaction assembly may be subjected to high-temperature treatment, vibration, ultrasound, etc. by combining an additional incubator, which assists in lysing the test sample. After the test sample is fully lysed, the deformable assembly and the breakable assembly are pressed from one side, such that the breakable assembly is broken from the bottom, and a channel is formed on the blocking assembly. Therefore, an upper area and a low area of the test cartridge are in communication with each other. The reagents in the deformable assembly and the reaction assembly can be mixed by repeatedly pressing the deformable assembly or inverting the test cartridge. After the reagents are fully mixed, the test cartridge is placed upright, and the liquids flow to the bottom of the reaction assembly. The flow of the liquids can be accelerated with the assistance of an operation of pressing the deformable assembly. Thereafter, the bottom of the test cartridge is heated through the incubator, such that the reagents are subjected to a nucleic acid amplification reaction. Results of the amplification reaction may be interpreted through a fluorescence signal, a visible light signal, turbidimetry, nucleic acid lateral chromatography, etc.

### EXAMPLE THREE

The solution provides a test method of a multi-functional integrated molecular test cartridge. The test method achieves a molecular test by the multi-functional integrated molecular test cartridge described in Example One, and includes steps as follows:
pre-load a multi-purpose buffer into a reaction assembly 30, and pre-load a reaction reagent into a deformable assembly 10;
apply an external force to the deformable assembly 10 to switch a breakable assembly 40 to a broken state;
apply an external force repeatedly to the deformable assembly 10 or invert the test cartridge, such that reagents in the reaction assembly 30 and the deformable assembly 10 are mixed; and
place the test cartridge upright after the reagents are fully mixed, such that all liquid flows to a bottom of the reaction assembly 30, add a test object into the reaction assembly 30, and place the reaction assembly 30 in an incubator for amplification.

It should be supplemented that the amplification reaction mentioned in Example Two or Example Three includes PCR amplification reaction, and further includes isothermal amplification reactions such as CPA, RPA, LAMP and RCA, and the multi-functional integrated molecular test cartridge provided in the solution can be suitable for tests for sputum, pus and blood.

In order to verify an effect of the multi-functional integrated molecular test cartridge provided in the solution, the following examples were carried out for verification:

### Example b 1

A human β-actin gene and a ribonucleic acid (RNA) product thereof were detected through cross priming amplification-real time fluorescence.

Amplification primers and probes and an amplification system are as follows: A human β-actin gene primer for CPA is specifically as shown in Table 1.

TABLE 1
Components of human β-actin
gene primer system for CPA

| | Sequence | |
|---|---|---|
| Forward | 5′-AGTACCCCATCG | SEQ ID |
| peripheral | AGCACG-3′ | NO: 1 |
| primer | | |
| FB | | |
| Reverse | 5′-AGCCTGGATAGC | SEQ ID |
| peripheral | AACGTACA-3′ | NO: 2 |
| primer | | |
| RB | | |
| Forward | 5′-GAGCCACACGCAG | SEQ ID |
| cross | CTCATTGTATCACCAA | NO: 3 |
| amplification | CTGGGACGACA-3′ | |
| primer | | |
| CPF | | |
| Reverse | 5′-CTGAACCCCAAG | SEQ ID |
| cross | GCCAACCGGCTGGGG | NO: 4 |
| amplification | TGTTGAAGGTC-3′ | |
| primer | | |
| CPR | | |
| Enhanced | 5′-GAGTGTGGGTGT | SEQ ID |
| primer | TCCCTTTGTACG | NO: 5 |
| IP1 | GGCCCG-3′ | |
| Detection | 5′-(fluorescein | SEQ ID |
| probe | amidite | NO: 6 |
| IP2 | (FAM))-GCGTCGGC | |
| | CTACCCTCGTCCTAA | |
| | CACGGGAGCCTGCAC | |
| | TGACCCGACG | |
| | C-(BHQ1)-3′ | |

A CPA reaction system is specifically as shown in Table 2.

TABLE 2
CPA reaction system

| Component | Volume |
|---|---|
| β-actin-FB (20 μM) | 4 μl |
| β-actin-RB (20 μM) | 4 μl |
| β-actin-CPF (20 μM) | 20 μl |
| β-actin-CPR (20 μM) | 20 μl |
| β-actin-IP1 (20 μM) | 15 μl |
| β-actin-IP2 (20 μM) | 7 μl |
| Sample protective agent | 10 μl |
| Moloney murine leukemia virus (M-MLV) reverse | 1 μl |
| transcriptase | |
| Bacillus stearothermophilus (Bst) DNA polymerase | 1 μl |

According to the above volumes, enzymes, primers, probes, etc. required for reactions were made into a premixed solution for being prepared into freeze-dried balls through freeze-drying, and the freeze-dried balls were pre-loaded into a reaction assembly; and reaction materials (i. e. , 120 μl of a CPA buffer and 193 μl of double distilled water (ddH₂O)) required for reactions were pre-loaded into a deformable assembly.

Step 1: human pharyngeal oral epithelial cells were collected with a sterile swab.

Step 2: a deformable assembly and a breakable assembly were pressed from one side, such that the breakable assembly was broken from a bottom, and a small hole was formed on a blocking assembly; and the deformable assembly was pressed, such that a pre-loaded reaction material flowed into a reaction assembly to be fully mixed with a freeze-drying reagent.

Step 3: a test cartridge was unscrewed and opened from a middle, a head of the collected swab was inserted into a liquid for sample elution, and then the swab was taken out. This step completed elution of the epithelial cells, and a test sample was protected due to the action of a sample protective agent.

Step 4: the test cartridge was screwed and closed, and the test cartridge was placed on a thermostatic fluorescence incubator at 58° C. for nucleic acid amplification and test for 20 min.

Step 5: after a reaction was finished, whether a test result is positive or not was determined by measuring a difference between an initial fluorescence value and a final fluorescence value of the reaction.

Test results are as shown in Table 3.

TABLE 3
Test results

| | Fluorescence | |
|---|---|---|
| | difference (>500 | |
| Sample | means positive) | Test result |
| Human pharyngeal swab 1 | 2,045 | Positive |
| Human pharyngeal swab 2 | 1,952 | Positive |
| Human pharyngeal swab 3 | 1,780 | Positive |
| Human pharyngeal swab 4 | 1,965 | Positive |
| Human pharyngeal swab 5 | 2,135 | Positive |
| Human pharyngeal swab 6 | 1,795 | Positive |
| Human pharyngeal swab 7 | 2,039 | Positive |
| Human pharyngeal swab 8 | 1,896 | Positive |
| Human pharyngeal swab 9 | 2,257 | Positive |
| Human pharyngeal swab 10 | 1,893 | Positive |
| Normal saline swab 1 | 135 | Negative |
| Normal saline swab 2 | 124 | Negative |
| Normal saline swab 3 | 118 | Negative |
| Normal saline swab 4 | 190 | Negative |
| Normal saline swab 5 | 213 | Negative |
| Normal saline swab 6 | 156 | Negative |
| Normal saline swab 7 | 144 | Negative |
| Normal saline swab 8 | 189 | Negative |
| Normal saline swab 9 | 172 | Negative |
| Normal saline swab 10 | 138 | Negative |

Conclusion: The test results are consistent with sources of the samples. After amplification, all human samples have fluorescence differences of 1,780-2,257 and are positive. All non-human samples have fluorescence differences of 118-213 and are negative. This method can effectively detect the human B-actin gene and the RNA product thereof.

### Example 2

Mycobacteria were rapidly detected through cross priming amplification-fluorescent probe.

Reagents were pre-loaded through a method as follows: 600 μL of a sample processing solution was pre-loaded into a reaction assembly; a drying reagent required for a reaction was pre-loaded into a deformable assembly; the sample processing solution and the drying reagent were separated by a blocking assembly and a breakable assembly; and the reagents are subjected to a test through a cross priming amplification-fluorescent probe.

Components of the drying reagent in the deformable assembly are as shown in Table 4.

TABLE 4
Drying reagent

| Component | Volume |
|---|---|
| Glyceraldehyde-3-phosphate | 1.2 pmol |
| dehydrogenase (GAPDH)-FB | |
| GAPDH-RB | 1.2 pmol |
| GAPDH-CPF | 10 pmol |
| GAPDH-CPR | 10 pmol |
| GAPDH-IP1 | 3 pmol |
| GAPDH-IP2 | 1.2 pmol |
| 6110-FB | 1.1 pmol |
| 6110-RB | 1.1 pmol |
| 6110-CPF | 12 pmol |
| 6110-CPR | 12 pmol |
| 6110-IP1 | 4 pmol |
| 6110-IP2 | 1.2 pmol |
| Bst DNA polymerase | 100 U |
| MgSO₄ | 3 pmol |
| NH₃SO₄ | 10 pmol |
| Drying auxiliary reagent | 50 mg |

Sequences of primers and probes for a mycobacterium tuberculosis IS6110 fragment test system are as shown in Table 5.

TABLE 5
Sequences of primers and probes

| | Sequence | |
|---|---|---|
| Forward | 5′-TCAACCGG | SEQ ID |
| peripheral | GAGCCCAG-3′ | NO: 7 |
| primer | | |
| 6110-FB | | |
| Reverse | 5′-TTTGCCGC | SEQ ID |
| peripheral | GGGTGGTC-3′ | NO: 8 |
| primer | | |
| 6110-RB | | |
| Forward | 5′-CGTAAACA | SEQ ID |
| cross | CCGTAGTTGGC | NO: 9 |
| amplification | GGCGCGCGATG | |
| primer | GCGAACTCA-3′ | |
| 6110-CPF | | |
| Reverse | 5′-GTGCCCGC | SEQ ID |
| cross | AAAGTGTGGCT | NO: 10 |
| amplification | AACAGTTTGGT | |
| primer | CATCAGCCGTT | |
| 6110-CPR | C-3′ | |
| Enhanced | 5′-TGGACGCG | SEQ ID |
| primer | GCTGATGTGC-3′ | NO: 11 |
| 6110-IP1 | | |
| Detection | 5′-(FAM)-GC | SEQ ID |
| probe | GTCGGCCTGAA | NO: 12 |
| 6110-IP2 | CCGTGAGGGCA | |
| | TCGAGGACCCG | |
| | ACGC-(BHQ1)-3′ | |

Sequences of primers and probes for a human GAPDH gene internal standard detection system are shown in Table 6.

Sequences of primers and probes

| | Sequence | |
|---|---|---|
| Forward | 5′-GGGGAGGCG | SEQ ID |
| peripheral | TGTGTGT-3′ | NO: 13 |
| primer | | |
| GAPDH-FB | | |
| Reverse | 5′-CCCCATACGA | SEQ ID |
| peripheral | CTGCAAAGAC-3′ | NO: 14 |
| primer | | |
| GAPDH-RB | | |
| Forward | 5′-TGGTGTCTGA | SEQ ID |
| cross | GCGATGTGGCGCC | NO: 15 |
| amplification | ACTAGGCGCTCAC | |
| primer | T-3′ | |
| GAPDH-CPF | | |
| Reverse | 5′-AGGTCGGAGT | SEQ ID |
| cross | CAACGGGTGATCG | NO: 16 |
| amplification | TAGACGCGGTTCG | |
| primer | G-3′ | |
| GAPDH-CPR | | |
| Enhanced | 5′-CTGCGCGGAG | SEQ ID |
| primer | GGAGAGAA-3′ | NO: 17 |
| GAPDH-IP1 | | |
| Detection | 5′-(cyanine5 | SEQ ID |
| probe | (cy5))-GCGTC | NO: 18 |
| GAPDH-IP2 | GGCCGCCCTGG | |
| | GCTGCGACCCC | |
| | CGACGC- | |
| | (BHQ2)-3′ | |

Components of each liter of sample processing solution are shown in Table 7.

TABLE 7
Sample processing solution

| Component | Volume |
|---|---|
| Tris-HCl at PH 8.5 | 100 mM |
| Triton X100 | 1.5 mM |
| EDTA disodium salt | 1 mM |
| Sample lysis buffer | 1% |
| Nuclease-free water | Carry out replenishment to 1 |
| | L |

At first, 20 negative tongue swabs from healthy people were collected, a small amount of high-concentration mycobacterium bovis bacille Calmette-Guérin (BCG) was added into heads of the swabs, and sample concentrations finally obtained were as follows: 5 samples of 10,000 bacteria/swab, 5 samples of 1,000 bacteria/swab, 5 samples of 100 bacteria/swab, and 5 samples of 0 bacterium/swab.

Test steps were as follows:

A test cartridge was unscrewed and opened from a middle, a simulated sample swab was inserted into a sample processing solution in a reaction assembly for elution, and then the swab was taken out and discarded.

The test cartridge was screwed and closed, a test cartridge hard storage part was inserted downwards into an incubator at 95° C. for 10 min, and the sample was lysed and inactivated.

After the sample was slightly cooled, a breakable assembly was broken from a bottom by applying a lateral force, such that an upper space and a lower space of the test cartridge are in communication with each other. The test cartridge was inverted, and a deformable assembly was pressed several times, such that the liquid flowed into the deformable assembly and was fully mixed with a pre-loaded drying reagent.

The test cartridge was placed upright, and the deformable assembly was pressed several times, such that the liquid flowed to a bottom of the reaction assembly.

The test cartridge, of which the reaction assembly faces downwards, was inserted into an incubator at 63° C. for amplification for 20 min. A change in a fluorescence signal was detected by a fluorescence detection probe on the incubator.

After a reaction was finished, whether a test result is positive, negative or invalid was determined by detecting a difference between an initial fluorescence value and a final fluorescence value of the reaction. Test results are as shown in Table 8.

TABLE 8
Test results

| | | FAM | Cy5 | |
|---|---|---|---|---|
| | | fluorescence | fluorescence | |
| | | difference | difference | |
| | | (>500 means | (>500 | Test |
| Sample | Concentration | positive) | means positive) | result |
| Simulated | 10,000 | 2,214 | 2,305 | Positive |
| sample 1 | bacteria/swab | | | |
| Simulated | 10,000 | 2,317 | 1,980 | Positive |
| sample 2 | bacteria/swab | | | |
| Simulated | 10,000 | 2,098 | 1,998 | Positive |
| sample 3 | bacteria/swab | | | |
| Simulated | 10,000 | 2,134 | 2,175 | Positive |
| sample 4 | bacteria/swab | | | |
| Simulated | 10,000 | 2,154 | 2,249 | Positive |
| sample 5 | bacteria/swab | | | |
| Simulated | 1,000 | 2,013 | 2,184 | Positive |
| sample 6 | bacteria/swab | | | |
| Simulated | 1,000 | 1,981 | 1,923 | Positive |
| sample 7 | bacteria/swab | | | |
| Simulated | 1,000 | 1,906 | 1,884 | Positive |
| sample 8 | bacteria/swab | | | |
| Simulated | 1,000 | 2,003 | 2,011 | Positive |
| sample 9 | bacteria/swab | | | |
| Simulated | 1,000 | 1,998 | 2,184 | Positive |
| sample 10 | bacteria/swab | | | |
| Simulated | 100 | 1,897 | 2,305 | Positive |
| sample 11 | bacteria/swab | | | |
| Simulated | 100 | 1,906 | 2,293 | Positive |
| sample 12 | bacteria/swab | | | |
| Simulated | 100 | 1,920 | 2,174 | Positive |
| sample 13 | bacteria/swab | | | |
| Simulated | 100 | 1,902 | 2,141 | Positive |
| sample 14 | bacteria/swab | | | |
| Simulated | 100 | 211 | 2,075 | Positive |
| sample 15 | bacteria/swab | | | |
| Simulated | 0 bacterium/swab | 186 | 2,203 | Negative |
| sample 16 | | | | |
| Simulated | 0 bacterium/swab | 175 | 2,198 | Negative |
| sample 17 | | | | |
| Simulated | 0 bacterium/swab | 164 | 2,245 | Negative |
| sample 18 | | | | |
| Simulated | 0 bacterium/swab | 153 | 2,174 | Negative |
| sample 19 | | | | |
| Simulated | 0 bacterium/swab | 138 | 2,305 | Negative |
| sample 20 | | | | |

The test results showed that all positive samples could be detected, and detection sensitivity of this method for simulated samples of mycobacterium bovis BCG could reach 100 bacteria/swab .

### Example 3

Specific fragments of nucleic acids of coronavirus disease 2019 were detected through cross priming amplification matching a nucleic acid chromatography test strip.

Amplification primers and probes and an amplification system are as follows:

Gene primers of a coronavirus disease 2019 test system are specifically as shown in Table 9.

Components of gene primers of
coronavirus disease 2019 test
system

| | Sequence | |
|---|---|---|
| Forward | 5′-TTCTTAGGAAT | SEQ ID |
| peripheral | CATCACAACTG-3′ | NO: 19 |
| primer | | |
| FB | | |
| Reverse | 5′-ATATCGATGTA | SEQ ID |
| peripheral | CTGAATGGGT-3′ | NO: 20 |
| primer | | |
| RB | | |
| Forward | 5′-GACACGGGTCA | SEQ ID |
| cross | TCAACTACATATGC | NO: 21 |
| amplification | ACCAAGAATGTAG | |
| primer | TTTACAGTC-3′ | |
| CPF | | |
| Reverse | 5′-GAGTAGGAGCT | SEQ ID |
| cross | AGAAAATCAGCACG | NO: 22 |
| amplification | ATTTAGAACCAGCC | |
| primer | TCATCC-3′ | |
| CPR | | |
| Detection | 5′-(FITC)-GACA | SEQ ID |
| probe | CGGGTCATCAACTA | NO: 23 |
| IP1 | CATATG-3′ | |
| Detection | 5′-(BIOTIN)-GA | SEQ ID |
| probe | GTAGGAGCTAGAAA | NO: 24 |
| IP2 | ATCAGCAC-3′ | |

An isothermal amplification reaction system is specifically as shown in Table 10.

TABLE 10
Isothermal amplification reaction system

| Component | Volume |
|---|---|
| FB (100 μM) | 0.2 μl |
| RB (100 μM) | 0.2 μl |
| CPF (100 μM) | 1 μl |
| CPR (100 μM) | 1.5 μl |
| IP1 (100 μM) | 0.3 μl |
| IP2 (100 μM) | 0.3 μl |
| Sample protective agent | 1 μl |
| M-MLV reverse transcriptase | 0.1 μl |
| Bst DNA polymerase | 0.1 μl |

According to the above volumes, enzymes, primers, probes, etc. required for reactions were made into a premixed solution, for being prepared into freeze-dried balls through freeze-drying, and the freeze-dried balls were pre-loaded into a reaction assembly;

and reaction materials (i.e. , 4 μl of a CPA buffer and 40 μl of ddH₂O) required for reactions were pre-loaded into a deformable assembly. During a reaction, two probes and primers containing fluorescein isothiocyanate (FITC) and BIOTIN groups respectively were connected to an amplification product by DNA polymerase. Line T of a nucleic acid lateral chromatography test strip specifically captured a product containing FITC and BIOTIN groups, and a control substance was added to a conjugate pad, and this substance could be captured at line C.

Step 1: a deformable assembly and a breakable assembly were pressed from one side, such that the breakable assembly was broken from a bottom, and a small hole was formed on a blocking assembly; and the deformable assembly was pressed, such that a pre-loaded liquid flowed into a reaction assembly to be fully mixed with a freeze-drying reagent.

Step 2: a test cartridge was unscrewed and opened from a middle, and coronavirus disease 2019 pseudovirus containing a detection fragment was added.

Step 3: the test cartridge was screwed and closed, and placed on a thermostatic fluorescence incubator at 58° C. for nucleic acid amplification and test for 20 min.

Step 4: after a reaction was finished, the test cartridge was inserted into a test cassette containing a sharp nucleic acid immunochromatographic test strip; and a bottom of the test cartridge was cut with a blade, such that a reaction product flowed onto the test strip, and a final result was obtained. Test results are shown in Table 11.

TABLE 11
Test results

| Sample | Line T | Line C | Result |
|---|---|---|---|
| 1,000 copies/reaction | +++ | ++ | Positive |
| 1,000 copies/reaction | +++ | +++ | Positive |
| 1,000 copies/reaction | ++ | ++ | Positive |
| 300 copies/reaction | +++ | +++ | Positive |
| 300 copies/reaction | ++ | +++ | Positive |
| 300 copies/reaction | ++ | +++ | Positive |
| 100 copies/reaction | ++ | +++ | Positive |
| 100 copies/reaction | + | ++ | Positive |
| 100 copies/reaction | ++ | ++ | Positive |
| 0 copy/reaction | − | +++ | Negative |
| 0 copy/reaction | − | +++ | Negative |
| 0 copy/reaction | − | + | Negative |

Note:
Different numbers of “+” from less to more indicate that intensity of a line is from weak to strong, and “−” indicates no line.

The results showed that sensitivity of this method for testing a coronavirus disease 2019 pseudovirus sample could reach 100 copies/reaction, and there is no false positive caused by non-specific amplification.

Those skilled in the art should understand that various technical features of the above examples can be arbitrarily combined. In order to simplify description, not all possible combinations of the various features of the above examples are described. However, if only the combinations of these technical features do not conflict, they should be considered to fall within the scope of the description of the present disclosure.

The above examples merely illustrate a plurality of embodiments of the present disclosure and are described specifically and detailedly, but cannot be construed as limiting the scope of the present disclosure. It should be noted that several variations and improvements can be made by those of ordinary skill in the art without departing from the concept of the present disclosure, and should all fall within the scope of protection of the present disclosure. Therefore, the scope of protection of the present disclosure should be subject to the appended claims.

What is claimed is:

1. A multi-functional integrated molecular test cartridge, suitable for testing a sample such as sputum, pus and blood, and comprising a deformable assembly (10), a blocking assembly (20) and a reaction assembly (30), wherein the blocking assembly (20) is placed between the deformable assembly (10) and the reaction assembly (30); the blocking assembly (20) and an outer wall of the deformable assembly (10) delimit a material storage area (11) for hermetically storing a material; a breakable assembly (40) is arranged on the blocking assembly (20), and the breakable assembly (40) is switched from a sealed state to a broken state under action of an external force; when the breakable assembly (40) is in the sealed state, the blocking assembly (20) blocks communication between the reaction assembly (30) and the deformable assembly (10); when the breakable assembly (40) is in the broken state, the breakable assembly (40) is separated from the blocking assembly (20), and a channel in communication with the reaction assembly (30) and the deformable assembly (10) is formed on the blocking assembly (20); a multi-purpose buffer is pre-loaded into the reaction assembly (30), the multi-purpose buffer is used for lysing or processing the sample and used as a buffer in an amplification reaction system, and the multi-purpose buffer is made into a dry material through vitrification, freeze-drying and volatilization-drying; a liquid or solid reaction reagent is pre-loaded into the deformable assembly (10); a test sample is added into the reaction assembly (30) and lysed by the multi-purpose buffer; after the test sample is completely lysed, an external force is applied to the deformable assembly (10) to switch the breakable assembly (40) to the broken state; and an external force is repeatedly applied to the deformable assembly (10) or the test cartridge is inverted, such that reagents in the reaction assembly (30) and the deformable assembly (10) are mixed

2. A multi-functional integrated molecular test cartridge, suitable for testing a sample such as sputum, pus and blood, and comprising a deformable assembly (10), a blocking assembly (20) and a reaction assembly (30), wherein the blocking assembly (20) is placed between the deformable assembly (10) and the reaction assembly (30); the blocking assembly (20) and an outer wall of the deformable assembly (10) delimit a material storage area (11) for hermetically storing a material; a breakable assembly (40) is arranged on the blocking assembly (20), and the breakable assembly (40) is switched from a sealed state to a broken state under action of an external force; when the breakable assembly (40) is in the sealed state, the blocking assembly (20) blocks communication between the reaction assembly (30) and the deformable assembly (10); when the breakable assembly (40) is in the broken state, the breakable assembly (40) is separated from the blocking assembly (20), and a channel in communication with the reaction assembly (30) and the deformable assembly (10) is formed on the blocking assembly (20); a multi-purpose buffer is pre-loaded into the reaction assembly (30), the multi-purpose buffer is used for lysing or processing the sample and used as a buffer in an amplification reaction system, and the multi-purpose buffer is made into a dry material through vitrification, freeze-drying and volatilization-drying; a liquid or solid reaction reagent is pre-loaded into the deformable assembly (10); an external force is applied to the deformable assembly (10) to switch the breakable assembly (40) to the broken state; an external force is repeatedly applied to the deformable assembly (10) or the test cartridge is inverted, such that reagents in the reaction assembly (30) and the deformable assembly (10) are mixed; and after the reagents are fully mixed, the test cartridge is placed upright, such that all liquid flows to a bottom of the reaction assembly (30), a test object is added into the reaction assembly (30), and the reaction assembly (30) is placed in an incubator for amplification.

3. The multi-functional integrated molecular test cartridge according to claim 1, wherein a first connecting area (13) is provided on an end side of the deformable assembly (10) close to the reaction assembly (20), a second connecting area (31) is provided on an end side of the reaction assembly (30) close to the deformable assembly (10) correspondingly, and the first connecting area (13) and the second connecting area (31) match each other, such that the deformable assembly (10) and the reaction assembly (30) are detachably connected.

4. The multi-functional integrated molecular test cartridge according to claim 2, wherein a first connecting area (13) is provided on an end side of the deformable assembly (10) close to the reaction assembly (20), a second connecting area (31) is provided on an end side of the reaction assembly (30) close to the deformable assembly (10) correspondingly, and the first connecting area (13) and the second connecting area (31) match each other, such that the deformable assembly (10) and the reaction assembly (30) are detachably connected.

5. The multi-functional integrated molecular test cartridge according to claim 1, wherein a channel penetrating the reaction assembly (30) and the deformable assembly (10) is arranged on the blocking assembly (20), the breakable assembly (40) is arranged at a position of the channel to block the channel, and when the breakable assembly (40) is separated from the blocking assembly (20), the channel on the blocking assembly (20) is exposed.

6. The multi-functional integrated molecular test cartridge according to claim 2, wherein a channel penetrating the reaction assembly (30) and the deformable assembly (10) is arranged on the blocking assembly (20), the breakable assembly (40) is arranged at a position of the channel to block the channel, and when the breakable assembly (40) is separated from the blocking assembly (20), the channel on the blocking assembly (20) is exposed.

7. The multi-functional integrated molecular test cartridge according to claim 4, wherein a main body of the breakable assembly (40) is a hard-material member, a joint between the breakable assembly (40) and the blocking assembly (20) is a soft-material member, a material of the soft-material member is easier to break than that of the hard-material member, and the breakable assembly (40) is broken by bending the deformable assembly to one side, and pressing or twisting the deformable assembly.

8. The multi-functional integrated molecular test cartridge according to claim 1, wherein the blocking assembly (20) is made of a breakable material, the breakable assembly (40) is made of a rigid material, the breakable assembly (40) is connected to a top of the blocking assembly (20), and when the breakable assembly (40) is subjected to the external force, the breakable assembly (40) is broken from a bottom, and a through hole is formed on the blocking assembly (20).

9. The multi-functional integrated molecular test cartridge according to claim 6, wherein the bottom of the breakable assembly (40) is a piercing member made of a hard material, when the breakable assembly (40) is in the sealed state, the piercing member is embedded into the blocking assembly (20), and when the breakable assembly (40) is in the broken state, the piercing member travels under the action of the external force, such that the channel in communication with the deformable assembly (10) and the reaction assembly (30) is formed on the blocking assembly (20).

10. The multi-functional integrated molecular test cartridge according to claim 1, wherein components of the multi-purpose buffer comprise: Tris-HCl at pH 6-9, dimethyl sulfoxide, TritonX 100, ethylenediaminetetraacetic acid (EDTA) disodium salt and dodecyl-β-D-maltoside.

11. The multi-functional integrated molecular test cartridge according to claim 2, wherein components of the multi-purpose buffer comprise: Tris-HCl at pH 6-9, dimethyl sulfoxide, TritonX 100, ethylenediaminetetraacetic acid (EDTA) disodium salt and dodecyl-β-D-maltoside.

12. A multi-functional integrated molecular test system, comprising:
the multi-functional integrated molecular test cartridge according to claim 1 and an incubator matching the test cartridge, wherein the multi-functional integrated molecular test cartridge is subjected to any one of high-temperature heating, vibration, ultrasound and amplification on the incubator.

13. A multi-functional integrated molecular test system, comprising:
the multi-functional integrated molecular test cartridge according to claim 2 and an incubator matching the test cartridge, wherein the multi-functional integrated molecular test cartridge is subjected to any one of high-temperature heating, vibration, ultrasound and amplification on the incubator.
